(19) United States
(12) Patent Application Publication
(10) Pub. No.: US 2009/0076347 A1
(43) Pub. Date: Mar. 19, 2009

(54) Pattern Recognition System for Classifying the Functional Status of Patients with Chronic Disease

(75) Inventors: Stephen T. Anderson, North Oaks, MN (US); Dean J. MacCarter, Englewood, CO (US); Ross Arena, Providence Forge, VA (US)

Correspondence Address: NIKOLAI & MERSEREAU, P.A., 900 SECOND AVENUE SOUTH, SUITE 820, MINNEAPOLIS, MN, 55402, US

(73) Assignee: SHAPE MEDICAL SYSTEMS, INC., North Oaks, MN (US)

(21) Appl. No.: 12/209,376

(22) Filed: Sep. 12, 2008

Related U.S. Application Data
(60) Provisional application No. 60/993,998, filed on Sep. 17, 2007.

(51) Int. Cl. A61B 5/00

(52) U.S. Cl. 600/301

(57) ABSTRACT

A method employing pattern recognition techniques for identifying the functional status of patients with chronic disease is described. This method describes a process by which sets of cardiopulmonary exercise gas exchange variables are measured during rest, exercise and recovery and stored as unique data sets. The data sets are then analyzed by a series of feature extraction steps, yielding a multi-parametric index (MPI) which reflects the current functional status of a patient. The method also employs a description scheme that provides a graphical image that juxtaposes the measured value of MPI to a reference classification system. An additional description scheme provides a trend plot of MPI values measured on a patient over time to provide feedback to the physician on the efficacy of therapy provided to the patient. The method will enable physicians to gather, view, and track complicated data using well-understood visualization techniques to better understand the consequences of their therapeutic actions.

### CROSS-REFERENCED TO RELATED APPLICATIONS

This application is a non-provisional application of Application No. 60/993,998, filed Sep. 17, 2007 and claims priority from that application which is also deemed incorporated by reference in its entirety in this application.

### STATEMENT REGARDING FEDERALLY SPONSORED RESEARCH OR DEVELOPMENT

Not applicable

### BACKGROUND OF THE INVENTION

I. Field of the Invention

The present invention relates generally to the field of medical diagnosis and specifically to a process of classifying a patient's functional status to assess the severity of the patient's disease. The disclosed method provides a more sensitive method that is easier to use than currently available classification systems. In addition, the present invention provides feedback during long-term follow-up in patients with chronic diseases.

II. Related Art

Current classification systems include those formulated by the New York Heart Association (NYHA) and by Dr. Karl Weber. The NYHA system places patients in one of four categories based on how much they are limited during physical activity:

| Class | Patient Symptoms |
|---|---|
| Class I | No limitation of physical activity. Ordinary physical |
| (Mild) | activity does not cause undue fatigue, palpitation, or |
| | dyspnea (shortness of breath). |
| Class II | Slight limitation of physical activity. Comfortable at |
| (Mild) | rest, but ordinary physical activity results in |
| | fatigue, palpitation, or dyspnea. |
| Class III | Marked limitation of physical activity. Comfortable at |
| (Moderate) | rest, but less than ordinary activity causes fatigue, |
| | palpitation, or dyspnea. |
| Class IV | Unable to carry out any physical activity without |
| (Severe) | discomfort. Symptoms of cardiac insufficiency at rest. If |
| | any physical activity is undertaken, discomfort is increased. |

The Weber classification system is a well established method for categorizing patients into four classes according to peak oxygen consumption or anaerobic threshold.

A major shortcoming of the NYHA system is that it relies on subjective observations by the patient and interpretation of those observations by the physician. Recent scientific literature has identified several flaws in the Weber system, including, for example:
(1) Peak VO₂ may be lower than maximally possible (does not represent a true max). A. Gitt, Circulation 2002; 106: p 3079-3084
(2) Sub maximal parameters are more practical than peak VO₂, and a more appropriate predictive index. M. Hollenberg, Journal of American College of Cardiology; 2000; 36: p 197-201

Traditionally, maximal cardiopulmonary exercise testing is performed in patients with heart failure as well as other chronic diseases to estimate functional capacity, test for ischemia and to follow general health status. Cardiopulmonary exercise testing is also used in this population clinically to follow response to treatment such as adding new medications, titrating medications, or device therapy. This form of testing is expensive and requires a medical team including MD supervision, RN or exercise specialists, along with a technician to perform the exercise studies. In addition, the equipment necessary includes a number of independent devices including an EKG system which is often integrated into a treadmill or stationary bike, metabolic cart, and a separate oximetry system. Maximal exercise testing is also a test that patients don't look forward to performing, and with heavy exercise there are increased risks.

There is a wealth of literature demonstrating the prognostic value of cardiopulmonary exercise testing, primarily in patients diagnosed with heart failure. (1) Several variables have demonstrated prognostic value including aerobic capacity (2), ventilatory efficiency (3, 4), end tidal carbon dioxide (5) and heart rate recovery (6). While the value of information garnered from this assessment technique is clear, clinical interpretation is presently cumbersome, limiting utilization of the cardiopulmonary exercise test. A formula that included all relevant exercise test variables, appropriately weighted according to prognostic value, and generating a single score would certainly improve clinical interpretation.

The importance of using a multiparametric approach to improving risk stratification has been reported in the literature (7). This article, however, only provides, the receiver operating characteristic curves of three sequential multivariate proportional hazard models. No means are provided to utilize this information in a clinical setting—only the ROC curves are provided, leaving it to the physician to interpret the meaning of multiple CPX, neurohormonal, and echo measurements.

The type of exercise (treadmill, bike, stepping) can influence the statistical interpretation of data set. In other words, a slope variable measured during a stair step exercise as shown in FIG. 2 will not necessarily have the same value when measured using a ramped cycle ergometer.

Previously, cardiopulmonary measurements have been made using discrete stages (e.g. Bruce protocol) or ramped protocols that continue until patient symptoms (exhaustion) occur, at which point the test is terminated. The present invention contemplates a simple three-step test (rest, exercise, recovery) which makes use of resting values, average values of exercise measurements, and their difference for multiparametric consideration.

An earlier method used the scientific literature (single source) derived mean value, standard deviation, and a normalizing value (NV), (“the number of Standard Deviations used to define the normal distribution) to calculate a variable called Autononic Balance Index. The NV was used to calculate an ABI coin and the NV was usually set to 2, since this is the classically defined definition of the “normal” range of values for a population measurements”.

### SUMMARY OF THE INVENTION

In contrast, the present method utilizes two values obtainable independently from the literature—normal value and cutoff point. These and the measured slope (or difference value) are inserted into the equation: 1+((measured value−normal value)/cutoff point−normal value). The above equation results in a negative value when the measured value is incrementally beyond the cutoff point, and the computation yields a number that is similar in magnitude for large or small values of normal and cutoff for individual parameters.

The present method insures that truly submaximal protocols can be used to produce valid clinical results. and avoids the need for peak testing to achieve the desired result. The present invention further teaches a method for determining cutoff points retrospectively from disease specific data sets, thereby insuring clinical validity of the multparametric calculation.

Thus, the present invention, to a large extent, obviates the problems discussed in the foregoing for each of the systems and utilizes the submaximal parameters that improve the predictive power over that of peak VO₂ alone. In the present invention, a continuous, numeric multiparametric ranking score or index (MPI) is used to provide an easier to visualize and interpret functional classification for heart disease patients. As indicated, this multiparametric score does not require exercising the patient to a maximal value, but, instead, utilizes gas exchange variables commonly measured during submaximal exercise. While maximal testing will still be required for patients with expected ischemia, a formulaic combination of submaximally obtained variables and peak VO₂ will improve clinical interpretation for this population as well.

The literature increasingly has begun to support the idea that a number of gas exchange variables commonly measured during submaximal exercise may be as good or better predictors of general health status and prognosis than values obtained during peak or maximal levels of activity. For example, it is known that ventilation relative to carbon dioxide production (V_{E}/VCO₂) within the first few minutes of exercise is highly predictive of death and is as much or more predictive than peak oxygen consumption. The link between cardiac function and respiratory gas exchange is likely related to high filling pressures which are transferred back to the pulmonary circulation stimulating breathing and altering gas exchange. Thus, other non invasive variables will also change, including the oxygen uptake efficiency slope (VO₂/log VE), Chronotropic Response Index (CRI), heart rate recovery, O₂ Pulse (VO₂/HR), end tidal CO₂ values (PetCO₂), and breathing pattern (e.g., breathing frequency, fb, and tidal volume, V_{T}, as well as an index of lung compliance, the slope of fb vs carbon dioxide production, VCO₂). Thus, it has been found that with worsening disease states, gas exchange will change in parallel, and these changes can form the basis for long term monitoring of the patient's functional status. Based on the above, an individualized set of parameters is selected to be followed.

Outcome Measurement: After the individualized set of parameters optimally is selected as described, the next step is to make an overall assessment of the patient's functional status over time. In order to appropriately assess the patient's functional status that is, in turn, related closely to adverse patient outcomes, the patient must be stressed, but only normally by mild to moderate exercise, in order to evaluate changes in the sympathetic and parasympathetic components of autonomic balance during dynamic, isotonic exercise and recovery. In other words, a volume load must be placed on the heart in order to assess the cardiopulmonary system's true response to patient activity. It should be noteworthy that it is the same approach with the assessment of cardiac ischemia using the classical ECG stress test. That is, some type of exercise modality must be used in order to stress the heart and create an imbalance in myocardial oxygen supply and demand. Unlike the classical ECG stress test, however, maximal exercise intensity is unnecessary to obtain the measured data. Instead, exercise intensities that reflect those normally experienced by the patient's activities of daily living are used to provide the volume load.

### ADVANTAGES

In one study (8), symptom limited CPX tests were performed in 127 patients (age 62.2±14). Anaerobic threshold (AT), determined by the Wasserman “V” slope method, was used for Weber classification. Ventilatory efficiency was derived using sub-maximal exercise data sets by the sub-max linear regression slope of VE/VCO₂. Oxygen uptake efficiency was derived using sub-maximal exercise data sets by the sub-max linear regression slope of VO₂/log VE. The Chronotropic Response Index CRI was derived using sub-maximal exercise data sets by the sub-max linear regression slope of % heart rate reserve/% metabolic reserve (Wilkoff formula). MPI was derived using the above 3 CPX parameters. Percent change amongst Weber Classes was analyzed using MPI and VO₂ AT, further quantifying the degree of differentiation between Weber classification, and the results are shown in the following table.

| | | | | Weber |
|---|---|---|---|---|
| Select CPX | Weber A | Weber B/% Δ | Weber C/% Δ | D/% Δ |
| Parameters | (n = 43) | (n = 47) | (n = 45) | (n = 22) |
| VO₂ at AT | 17.7 ± 4.8 | 12.4 ± .1.0 | 9.5 ± .9 | 7.4 ± .5 |
| | | (30%) | (23.4%) | (22%) |
| MPI | +2.4 ± 2.3 | −.2 ± 2.0 | −2.3 ± 2.0 | −3.7 ± 1.4 |
| | | (108% ↓) | (1050% ↓) | (61% ↓) |
| “p” value | | p < .0001 | p < .0001 | p = .006 |

Although the MPI change from Weber A to B marked a + to − change in MPI value, the largest significant transition was observed between Weber classes B and C with further deterioration (> negative value) from Weber functional class C to D. The average % change or inter class discrimination between Weber classes using the cumulative MPI was 406%, as compared to 25% for VO₂ AT alone. It will be appreciated that the novel MPI score of the present invention offers a simplified, more sensitive, easier to interpret quantitative means for functional classification. In addition, this is accomplished in a manner that is less stressful to the patient.

### BRIEF DESCRIPTION OF THE DRAWINGS

In the drawings:

FIG. 1 is a schematic drawing that illustrates the functional components of a CPX testing system usable with the present invention;

FIG. 2 is a schematic drawing that illustrates one form of exercise protocol that is used to place a volume load on the cardiopulmonary system;

FIG. 3 illustrates the organization of the measured data once it is acquired from the cardiopulmonary exercise gas exchange analyzer;

FIG. 4 illustrates the table of data used in part to calculate the Ranking Parameter (RP) used in the present invention;

FIG. 5 illustrates the results of a regression analysis of two cardiopulmonary data pairs FIG. 6 illustrates the determination of a delta value for normal and diseased patients FIG. 7 illustrates the equations used to calculate the Ranking Parameters (RP);

FIG. 8 illustrates a description scheme employed by the present invention for displaying the resultant MPI value from a test with the Weber Class juxtaposed onto the scale;

FIG. 9 illustrates a trend plot of time-sequential MPI test values.

### DETAILED DESCRIPTION

The following detailed description with respect to patient data is intended to be exemplary of a preferred method of utilizing the concepts of the present invention and is not intended to be exhaustive or limiting in any manner with respect to similar methods and additional or other steps which might occur to those skilled in the art. The following description further utilizes illustrative examples, which are believed sufficient to convey an adequate understanding of the broader concepts to those skilled in the art, and exhaustive examples are believed unnecessary.

General Considerations

The present invention involves a pattern recognition system which includes data gathering, feature extraction and classification aspects. Data is taken by a cardiopulmonary exercise gas exchange analyzer that gathers observations to be classified or described. A feature extraction mechanism computes numeric information from the observations and a classification or description scheme accomplishes the actual job of classifying or describing observations based on the extracted features. These aspects will be described in greater detail.

Data Gathering

The general class of data utilized in the system of the present invention, cardiopulmonary exercise gas exchange measurements, is obtained 1) at rest, 2) during physical exercise testing performed in accordance with a standardized workload protocol as the forcing function to elicit physiologic changes resulting from the workload, and 3) during a short recovery period following exercise termination. Data measured during exercise quantifies how an individual is able to function in the physical world in terms of the physiologic changes that the individual experiences when engaged in the performance of daily physical work.

Physiologic changes are measured using a cardiopulmonary exercise testing system (CPX) to measure selected variables associated with oxygen consumption, VO₂, carbon dioxide production, VCO₂, end-tidal CO₂, PetCO₂, ventilation, VE, and heart rate, HR.

The data gathering aspect of the invention involves known techniques and analyses, and the calculations for formulating predictive assessments available, in some cases, in the scientific literature (see the bibliography in References). Importantly, it is aspects of the retrospective analysis of disease specific data sets, the feature extraction mechanism, and the classification scheme from which the invention enables an observer to gain new and valuable insight into the present condition and condition trends in patients. Thus, in accordance with a preferred method, a cardiopulmonary exercise gas exchange analysis is made for each test data set. The performance of such a test is well understood by individuals skilled in the art, and no further explanation of this is believed necessary.

Equipment

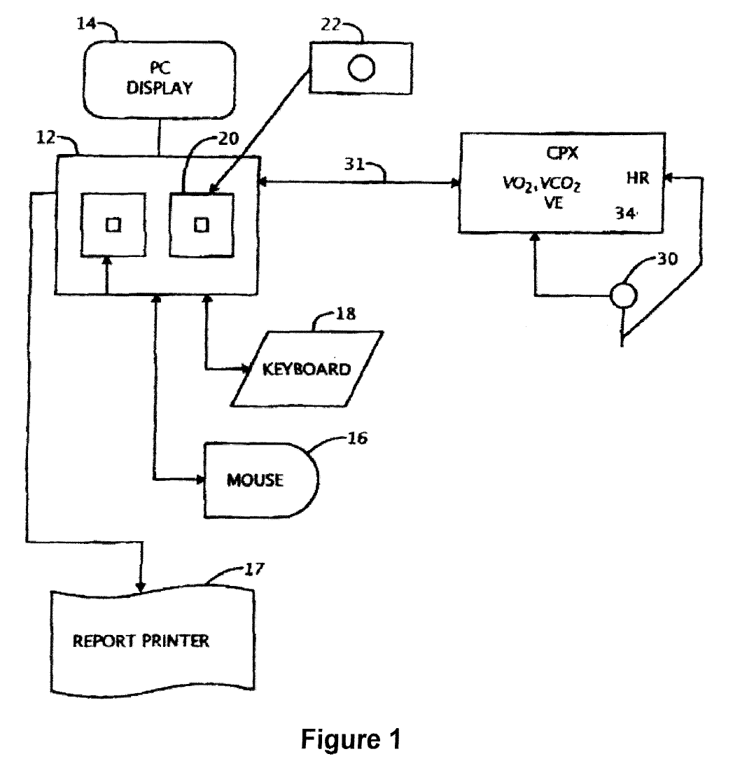

With this in mind typical hardware is shown in FIG. 1, which illustrates typical equipment whereby a cardiopulmonary exercise test (CPX) may be conducted and the results displayed in accordance with the method of the present invention. The system is seen to include a data processing device, here shown as a personal computer or PC 12, which includes a video display terminal 14 with associated mouse 16, report printer 17 and a keyboard 18. The system further has a compact disc handler 20 with associated compact disc 22. As is well known in the art, the compact disc handler 20 input/output interfaces comprise read/write devices for reading prerecorded information stored, deleting, adding or changing recorded information, on a machine-readable medium, i.e., a floppy disc, and for providing signals which can be considered as data or operands to be manipulated in accordance with a software program loaded into the RAM or ROM memory (not shown) included in the computing module 12.

The equipment used in the exercise protocol can be a simple stair step of a known height. A CPX testing system 34 interfaces with the subject 30 during operation of the exercise test. The physiological variables may be selected from heart rate (HR), ventilation (VE), rate of oxygen uptake or consumption (VO₂) and carbon dioxide production (VCO₂) or other variables derived from these basic measurements. Physiological data collected is fed into the computing module 12 via a conductor 31, or other communication device.

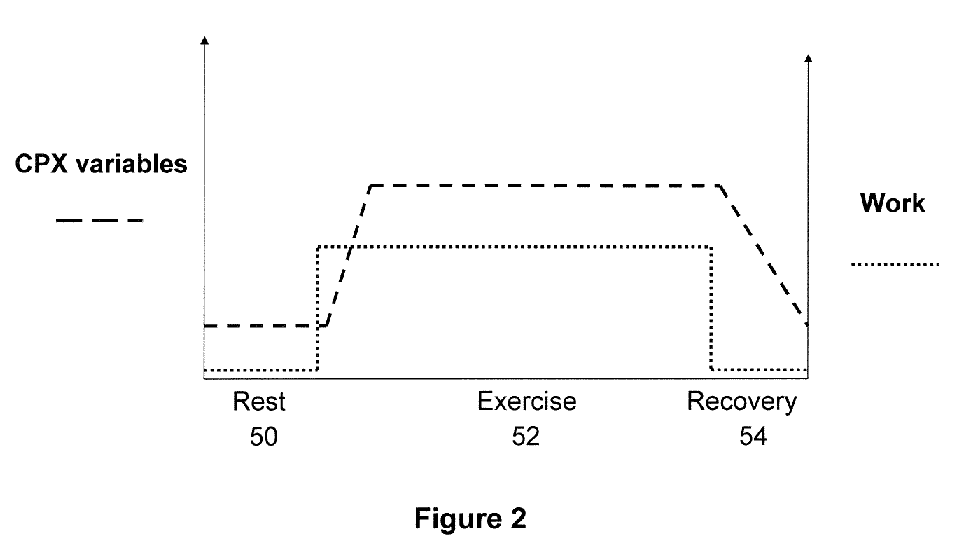

The workload protocol is illustrated in FIG. 2 and is organized into a rest phase 50, an exercise phase 52, and a recovery phase 54. Optionally, the workload may also be quantified by requiring the patient to maintain a desired stepping cadence by the addition of an audible metronome that guides the frequency of the steps taken during the exercise phase.

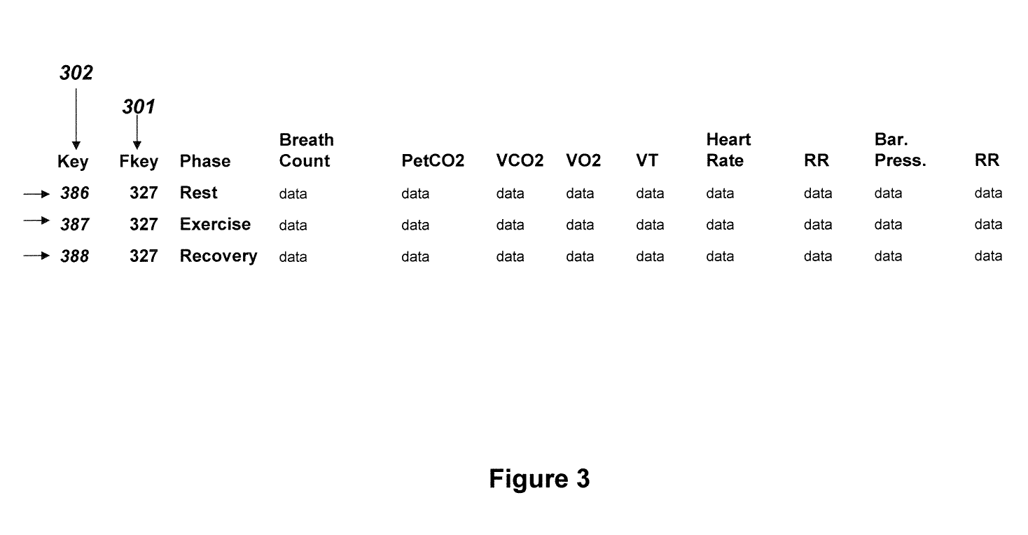

All data acquired by the CPX system may be stored in a relational database as illustrated in FIG. 3. Most importantly, data for each patient and each test (301) is stored into separate subsets of data (302) representing the rest phase 386, the exercise phase 387, and the recovery phase 388 for use by the feature extraction mechanism.

Feature Extraction

Two types of feature extraction are employed by the system of the present invention: 1) the slope of the line of regression obtained from select data pairs, 2) the difference between the average value of select variables or ratios of variables at rest and during exercise. Representative examples of each are:
1. Slopes—ventilation (VE) vs. carbon dioxide production (VCO₂), or ventilatory efficiency slope; oxygen uptake (VO₂) vs. log ventilation (VE), or oxygen uptake efficiency slope; % heart rate reserve vs. % metabolic reserve, or CRI; heart rate during one minute of recovery, or heart rate recovery.
2. Difference of average values at rest and during exercise—Partial pressure of end tidal CO2 (PetCO₂), Oxygen Pulse (VO₂/HR), dead space (VD) to tidal volume (VT), inspiratory drive (VT/Ti)

Feature Extraction—Step 1

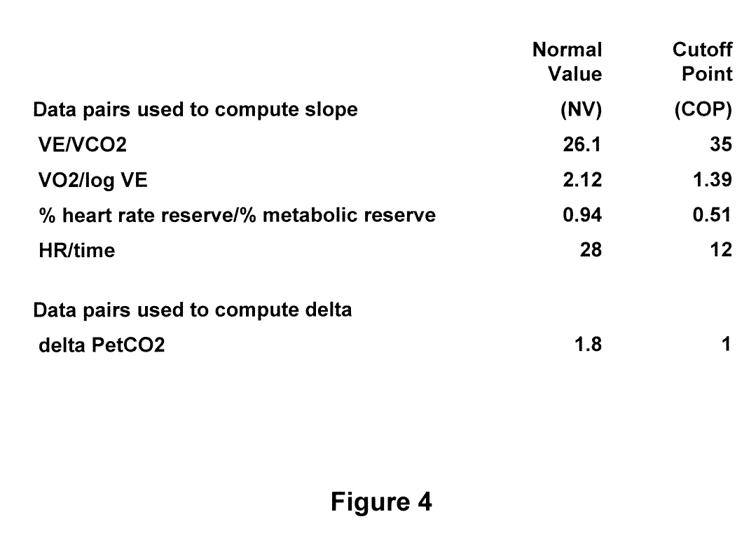

Support for the use of statistical pattern recognition also comes from new methods of analyzing cardiopulmonary data published in the scientific literature. From publications listed in the bibliography in the Reference section below, statistical values for the normal value and cutoff point can be obtained for each of the features extracted in Step 1 above. At the present time, these values only exist for the listed slope values, but future uses of such values for slope and for the difference and ratio classes are contemplated by the present invention. In FIG. 4, the available values for normal and cutoff point are stored in table form. It is anticipated that slight changes may be made to the values in FIG. 4 over time based on further studies.

Feature Extraction—Step 2a—Slopes

The next step is to compute the regression line through the select data pairs obtained from the database in FIG. 4. The general form for the regression equation is

y=a+bx

The constant a is the intercept, b is the slope. The a and b values are chosen so that the sum of squared deviations from the line is minimized. The best line is called the regression line, and the equation describing it is called the regression equation.

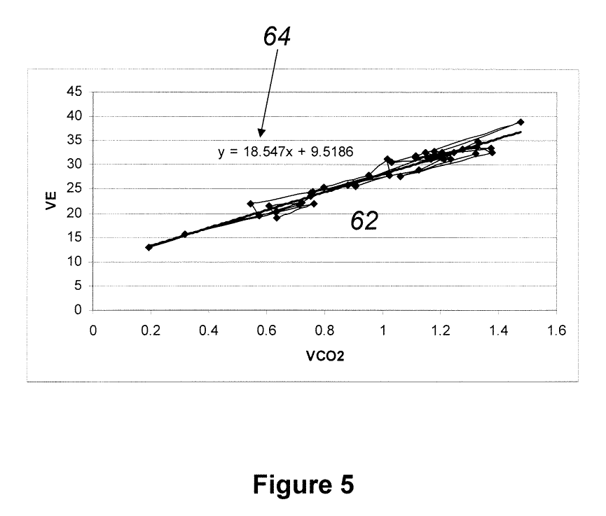

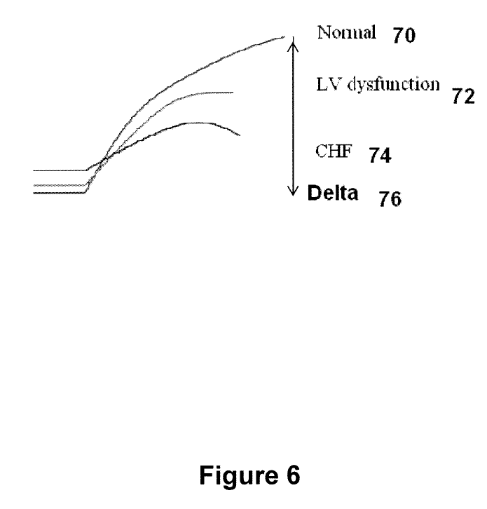

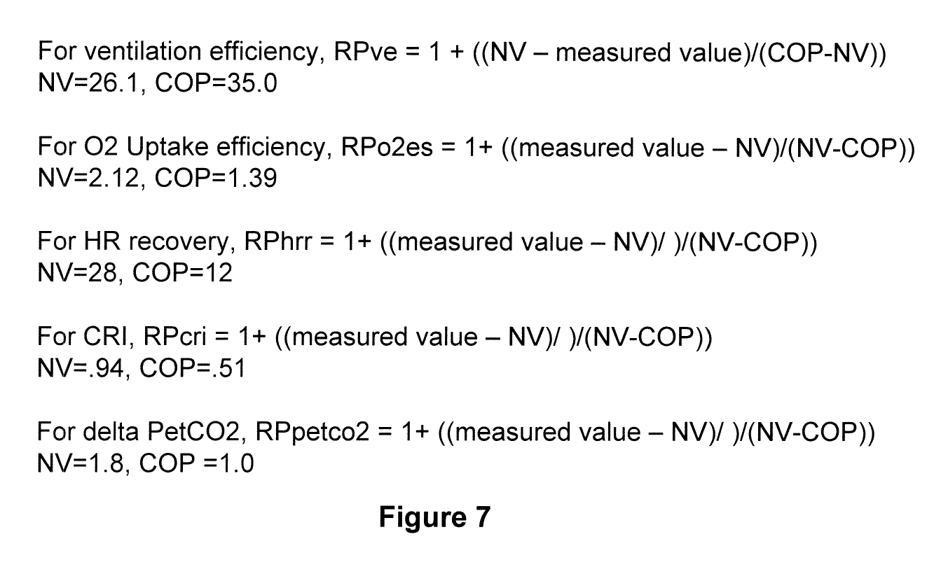

In FIG. 5, an example illustrates the measured data for the cardiopulmonary data pairs with the computed line of regression at 62, and the slope and correlation value shown at 64.

Feature extraction—Step 2B—Deltas

In FIG. 6, the idealized response to an increase in workload from the resting phase for O₂ pulse (VO₂/HR) is illustrated. The normal response is shown at 70, the response for a patient with left ventricle (LV) dysfunction is shown at 72, and the response for a patient with congestive heart failure (CHF) is shown at 74. The delta for the normal response 76 is indicated by the vertical line drawn from the resting value to the normal end of exercise value.

### Feature Extraction—Step 3

An individual ranking parameter (RP) is then computed for each of the select data pairs. The RP is calculated using the measured slope value, b, computed in Step 2a and the statistical values obtained from the clinical research or statistical analysis of disease specific data sets for the data pair and stored in the table in FIG. 4. A mitigating factor is that some variables (ventilatory efficiency slope) have high values indicating poor outcome. Some (oxygen uptake efficiency) have low values indicating poor outcome. For the case where “large is bad”, the first step is to subtract the measured value from the normal value (NV), or RPve=(NV−measured value)/(Cutoff Point−NV). For the case where “small is bad”, RPoeus=(measured value−NV)/(NV−Cutoff Point). By adding 1 to the above, the value of RP is forced to be 0 at a measured value that equals the Cutoff Point (COP). A set of formula equations for calculating various ranking parameters is shown in FIG. 7.

It has been arbitrarily decided that a negative value is undesirable. Thus, a negative RP indicates a poor outcome, a positive RP indicates a positive outcome. The more negative the RP value is, the greater the likelihood of a poor outcome.

### Feature Extraction—Step 4

The final step of feature extraction is to calculate the multiparametric index (MPI). The general form of the equation to do this is

MPI=W₁*RP₁+W₂*RP₂+ . . . +Wₙ*RPₙ

Where Wₙ=the weighting factor for the particular ranking parameter RPₙ.

Both RPₙ and Wₙ are determined by analyzing one or more large disease-specific datasets that include prognostic analysis for adverse-events. Univariate and multivariate Cox regression analysis will be performed to determine which cardiopulmonary exercise testing variables possess prognostic value. For this initial analysis, variables will be assessed as continuous variables. For the multivariate analysis, the forward stepwise method will be employed with entry and removal values set at 0.05 and 0.10, respectively. Receiver operating characteristic curve analysis will then be performed on variables retained in the multivariate regression to determine optimal dichotomous threshold values. Univariate Cox regression will then be employed again to determine the hazard ratios for dichotomous expressions of cardiopulmonary exercise testing variables retained in the multivariate regression. The defined hazard ratios can, optionally, be used as the weighting factors in the MPI. All statistical tests with a p-value <0.05 will be considered significant.

From this analysis, multiple versions of MPIs can be generated. For example, one will include both submaximal and maximal cardiopulmonary exercise test variables to be employed during symptom limited exercise testing. The other MPI would only include variables obtained during submaximal exercise to be used during testing procedures that do not bring a patient to maximal exertion.

Description Scheme—MPI Scale Plot

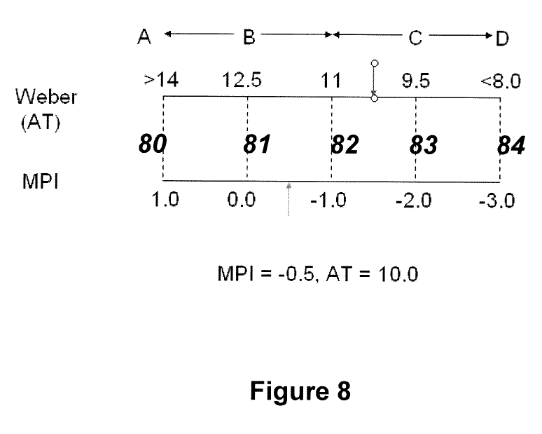

In order to provide a familiar frame of reference for physicians who use the classification system of the present invention, a preferred method for the description scheme is illustrated in FIG. 8. In the illustrated case, the Weber system utilizing anaerobic threshold is combined with a numerical scale for displaying the MPI value for the current patient test. The delineation between Weber classes and MPI values are shown at 80,81,82,83 and 84. Also illustrated is one example of how to display the calculated values of MPI and AT and their scale locations.

Description Scheme—Trend Plot

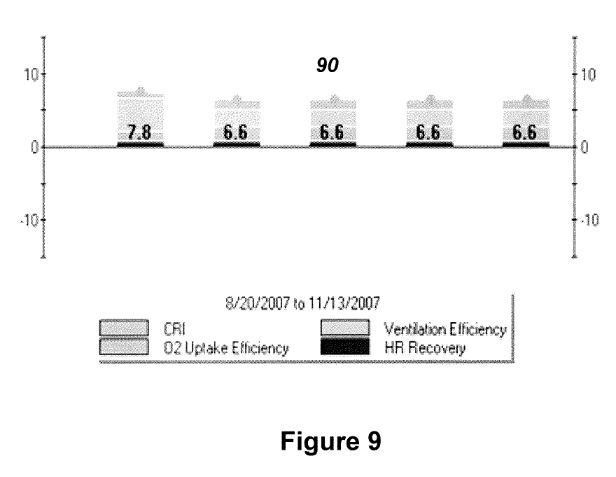

Of course, an important aspect of the value of the system of the present invention is the ability to provide a rapid assessment of the effect of any given therapy over time as by, for example, using a trend plot. One example of a trend plot for MPI value over time is illustrated in the bar chart in FIG. 9. In this example, the individual values of the RP are scaled and stacked to form a bar, and, in this manner, the MPI value determines the vertical height of each bar. The MPI value and date is then displayed in relationship to the bar. In this example, a line at 90 displays the numeric trend. However, it will be understood that there is no limitation intended in terms of the type of graph utilized or visual effects employed.

The invention has been described in considerable detail in order to comply with the Patent Statutes and to provide those skilled in the art with the information needed to apply the novel principles and to construct and use such specialized components as are required. However, it is to be understood that the invention can be carried out by specifically different equipment and devices, and that various modifications, both as the equipment details and operating procedures can be accomplished without departing from the scope of the invention itself.

### PUBLICATION REFERENCE LIST

1. Gibbons R J, Balady G J, Timothy B J, et al. ACC/AHA 2002 guideline update for exercise testing: summary article. A report of the American College of Cardiology/American Heart Association Task Force on Practice Guidelines (Committee to Update the 1997 Exercise Testing Guidelines). J Am Coll Cardiol 2002; 40:1531-40.
2. Arena R, Myers J, Williams M A, et al. Assessment of functional capacity in clinical and research settings: a scientific statement from the American Heart Association Committee on Exercise, Rehabilitation, and Prevention of the Council on Clinical Cardiology and the Council on Cardiovascular Nursing. Circulation 2007; 116:329-43.
3. Arena R, Myers J, Guazzi M. The clinical and research applications of aerobic capacity and ventilatory efficiency in heart failure: an evidence-based review. Heart Fail Rev 2008; 13:245-69.
4. Mancini D M, Eisen H, Kussmaul W, Mull R, Edmunds L H, Jr., Wilson J R. Value of peak exercise oxygen consumption for optimal timing of cardiac transplantation in ambulatory patients with heart failure. Circulation 1991; 83:778-86.
5. Arena R, Myers J, Abella J, et al. Development of a Ventilatory Classification System in Patients With Heart Failure. Circulation 2007; 115:2410-7.
6. Francis D P, Shamim W, Davies L C, et al. Cardiopulmonary exercise testing for prognosis in chronic heart failure: continuous and independent prognostic value from VE/VCO(2) slope and peak VO(2). Eur Heart J 2000; 21:154-61.
7. Scardozi A B, et al. Multiparametric Risk Stratification in Patients With Mild to Chronic Heart Failure. Journal of Cardiac Failure 2007; 13:445-51.

### MPI REFERENCES

### 1. VE Efficiency Slope:

Normal Values:
(a) 25.6±3.2; N=144;
Reference: Arena R, J Myers, J Abella, M A Peberdy, D Bensimhon, P Chase, M Gauzzi. Development of a ventilation classification system in patients with heart failure. Circulation, 2007;115:2410-2417.
(b) 26.2±4.0; N=101;
Reference: Kleber F X, G Vietzke, K D Wernecke, U Bauer, C Opitz, R Wensel, A Sperfeld, S Glaser. Impairment of ventilatory efficiency in heart failure: Prognostic impact. Circulation, 2000;101:2803-2809.
(c) 26.5±3.8; N=83;
Reference: Ponikowski P, D P Francis, M F Piepoli, L Ceri Davies, T P Chua, C H Davos, V Florea, W Banasiak, P A Poole-Wilson, A J S Coats, S D Anker. Enhanced ventilatory response to exercise in patients with chronic heart failure and preserved exercise tolerance: Marker of abnormal cardiorespiratory reflex control and predictor of poor prognosis. Circulation, 2001:103:967-972.
Mean normal value+26.1; N=328 subjects

Cut-Off Values for HF Patients:
(a) 35.0; N=600
Reference: Corra U, A Mezzani, E Bosimini, F Scapellato, A Imparato, P Giannuzzi. Ventilatory response to exercise improves risk stratification in patients with chronic heart failure and intermediate functional capacity. Am Heart J, 2002;143(3):418-426.
(b) 34.2 with ishemic disease and 34.5 with non-ischemic disease; N=268
Reference: Arena R, J Myers, J Abella, M A Peberdy. Influence of heart failure etiology on the prognostic value of peak oxygen consumption and minute ventilation/carbon dioxide production slope. Chest, 2005;128:2812-2817.
(c) 36.2; N=288
Reference: Guazzi M, R Arena, A Ascione, M Piepoli, M D Guazzi. Exercise oscillatory breathing and increased ventilation to carbon dioxide production slope in heart failure: An unfavorable combination with high prognostic value. Am Heart J, 2007;153:859-867.
Mean cut-off point=35.0; N=1156 HF patients
Note: The Ventilation Efficiency Classification reference by Arena and Meyers is also needed as a reference in the software. I believe you have this PDF file already (is in the reference list).

### 2. Oxygen Uptake Efficiency Slope (OUES)

Normal Values:
(a) 2.12±0.33; N=415;
Reference: A. Thomas McRae III, James B. Young, M L. Alkotob, Claire E. Pothier Snader, Eugene H. Blackstone, Michael S. Lauer. The Oxygen Efficiency Slope as a Predictor of Mortality in Chronic Heart Failure. J Amer. College Cardioolgy; Vol 43 (5) Suppl; 856-3.; 2002.
(b) 2.33±0.5 men; 1.60 women; N=998 total
Reference: M. Holenberg and Ira B. Tager. Oxygen Uptake Efficiency Slope: An Index of Exercise Performance and Cardiopulmonary Reserve Requiring Only Submaximal Exercise. J. Am Coll Cardiology; 2000;36:194-201.

Cut-Off Values for HF Patients:
(a) 1.4; N=341
Reference: Arena R, J Myers, L Hsu, M A Peberdy, S Pinkstaff, D Bensimhon, P Chase, M Vicenzi, M Guazzi. The minute ventilation/carbon dioxide production slope is prognostically superior to the oxygen uptake efficiency slope. J Cardiac Fail, 2007;13(6):462-469.
(b) 1.47; N=243
Reference: Davies L C, R Wensel, P Georgiadou, M Cicoira, A J S Coats, M F Piepoli, D P Francis. Enhanced prognostic value from cardiopulmonary exercise testing in chronic heart failure by non-linear analysis: oxygen uptake efficiency slope. E Heart J, 2006;27:684-690.
(c) 1.31; N=1245
Reference: A. Thomas McRae III, James B. Young, M L. Alkotob, Claire E. Pothier Snader, Eugene H. Blackstone, Michael S. Lauer. The Oxygen Efficiency Slope as a Predictor of Mortality in Chronic Heart Failure. J Amer. College Cardioolgy; Vol 43 (5) Suppl; 856-3.;2002.
Mean “Cut-off” point=1.39

### 3. Heart Rate Recovery

Normal Values:
(a) 33±9; N=4633
Reference: Watanabe J, M Thamilarasan, E H Blackstone, J D Thomas, M S Lauer. Heart rate recovery immediately after treadmill exercise and left ventricular systolic dysfunction as predictors of mortality: The case of stress echocardiography. Circulation, 2001;104:1911-1916.
(b) 23±8; N=2097
Reference: Deepak P. Vivekananthan, Eugene H.

Blackstone, Claire E. Pothier Snader, and Michael S. Lauer. Heart rate Recovery After Exercise Is a predictor of Mortality, Independent of the Angiographic Severity of Coronary Disease. J Am Coll Cardiology; 2003: 42:831-838.
Mean Normal Value=28±8.

Cut-Off Values for HF:
(a) < or =12; N=838
Reference: Deepak P. Vivekananthan, Eugene H.

Blackstone, Claire E. Pothier Snader, and Michael S. Lauer. Heart rate Recovery After Exercise Is a predictor of Mortality, Independent of the Angiographic Severity of Coronary Disease. J Am Coll Cardiology; 2003: 42:831-838.

### 4. CRI or Chronotropic Response Index

Normal Values:
(a) 0.94±0.16; N=470
Reference: Robbins M, G Francis, F J Pashkow, C E Snaker, K Hoercher, J B Young, M S Lauer. Ventilatory and heart rate responses to exercise: better predictors of heart failure mortality than peak oxygen consumption. Circulation, 1999;100:2411-2417.
(b) 0.93±0.15; N=323
Reference: Dresing T J, E H Blackstone, F J Pashkow, C E Snader, T H Marwick, S L Lauer. Usefulness of impaired chronotropic response to exercise as a predictor of mortality, independent of the severity of coronary artery disease. Am J Cardiol, 2000;86:602-609.

Cut-Off Values:
(a) HF patients: CRI=</=0.51
Reference: Robbins M, G Francis, F J Pashkow, C E Snaker, K Hoercher, J B Young, M S Lauer. Ventilatory and heart rate responses to exercise: better predictors of heart failure mortality than peak oxygen consumption. Circulation, 1999;100:2411-2417.
(b) Mild to severe/CAD: CRI</=0.8
Reference: Dresing T J, E H Blackstone, F J Pashkow, C E Snader, T H Marwick, S L Lauer. Usefulness of impaired chronotropic response to exercise as a predictor of mortality, independent of the severity of coronary artery disease. Am J Cardiol, 2000;86:602-609.

1. A method of pattern recognition for classifying the functional status of patients with chronic disease comprising characterizing said functional status based on a multiparametric index (MPI).

2. A method of pattern recognition for classifying the functional status of patients with chronic disease comprising characterizing said functional status based on a multiparametric index (MPI) wherein the MPI is computed using the equation
MPI=W₁*RP₁+W₂*RP₂+ . . . +Wₙ*RPₙ
Where RP=an individual Ranking Parameter and W=the weighting factor for the particular RP determined by either retrospective statistical analysis or by statistical analysis of patients groups prospectively over time.

3. A method as in claim 2 including at least one RP selected from the group consisting of ventilation efficiency, O₂ uptake efficiency, heart rate recovery, chronotropic response index (CRI) and delta end tidal CO₂ (Pet CO₂).

4. A method as in claim 2 wherein the value for RP is calculated, in part, from cardiopulmonary exercise test measurements.

5. A method as in claim 4 wherein the cardiopulmonary exercise test measurements are gathered from sub-maximal exercise bouts.

6. A method as in claim 3 wherein the value for RP is calculated, in part, from cardiopulmonary exercise test measurements.

7. A method as in claim 6 wherein the cardiopulmonary exercise test measurements are gathered from sub-maximal exercise bouts.

8. A method as in claim 4 wherein the cardiopulmonary exercise test measurements are gathered from maximal, symptom limited exercise bouts

9. A method as in claim 5 wherein cardiopulmonary exercise test measurements are displayed during low intensity exercise and stored as data sets, each set being associated with a rest phase, an exercise phase, and a recovery phase.

10. A method as in claim 7 wherein cardiopulmonary exercise test measurements are displayed during low intensity exercise and stored as data sets, each set being associated with a rest phase, an exercise phase, and a recovery phase.

11. A method as in claim 2 wherein the RP is determined, in part, by a feature extraction mechanism that computes, as the measured value, the slope of the line of regression obtained from select data pairs obtained in sub-maximal exercise bouts.

12. A method as in claim 3 wherein the RP is determined, in part, by a feature extraction mechanism that computes, as the measured value, the slope of the line of regression obtained from select data pairs obtained in sub-maximal exercise bouts.

13. A method as in claim 2 wherein the RP is determined, in part, by a feature extraction mechanism that computes, as the measured value, the difference between the average value of select variables or ratios of select variables obtained at rest and during exercise in sub-maximal exercise bouts.

14. A method as in claim 3 wherein the RP is determined, in part, by a feature extraction mechanism that computes, as the measured value, the difference between the average value of select variables or ratios of select variables obtained in sub-maximal exercise bouts at rest and during exercise.

15. A method as in claim 2 wherein the value for Ranking Parameter, RP, is calculated, in part, from known statistical values for the feature extraction mechanisms that compute a measured value selected from the group consisting of the slope of the line of regression obtained from select data pairs obtain in sub-maximal exercise bouts and the difference between the average value of select variables or ratios of selected variables obtained in sub-maximal exercise bouts.

16. A method as in claim 2 wherein the value for Ranking Parameter, RP, is calculated, in part, from retrospective analysis of disease specific data sets that include adverse-event data for the feature extraction mechanisms that compute a measured value selected from the group consisting of the slope of the line of regression obtained from select data pairs obtain in sub-maximal exercise bouts and the difference between the average value of select variables or ratios of selected variables obtained in sub-maximal exercise bouts.

17. A method as in claim 16 wherein univariate and multivariate Cox regression analysis is performed to determine whether new cardiopulmonary exercise testing variables possess prognostic value.

18. A method as in claim 17 wherein multivariate regression analysis using the forward stepwise method is employed with entry and removal values set at 0.05 and 0.10, respectively.

19. A method as in claim 17 wherein a receiver operating characteristic curve analysis is performed on variables retained in the multivariate regression to determine optimal dichotomous threshold values.

20. A method as in claim 19 wherein Univariate Cox regression analysis is employed again to determine the hazard ratios for dichotomous expressions of cardiopulmonary exercise testing variables retained in the multivariate regression.

21. A method as in claim 20 wherein the defined hazard ratios can optionally become the weighting factors in the MPI formula.

22. A method as in claim 12 wherein the statistical values include the normal value (NV) and cutoff point (COP).

23. A method as in claim 17 wherein the statistical values include the normal value (NV) and cutoff point (COP).

24. A method as in claim 2 wherein the RP is calculated using as inputs the parameters by a feature extraction mechanism that computes, as the measured value, the difference between the average value of select variables or ratios of select variables obtained at rest and during exercise in sub-maximal exercise bouts and applied to the formula RP=1+((NV−measured value)/(COP−NV)) for the case where a large measured value is predictive of poor outcome.

25. A method as in claim 2 wherein the RP is calculated using as inputs the parameters determined, in part, from known statistical values for the feature extraction mechanism that compute a measured value selected from the group consisting of the slope of the line of regression obtained from select data pairs obtain in sub-maximal exercise bouts and the difference between the average value of select variables or ratios of selected variables obtained in sub-maximal exercise bouts and applied to the formula RP=1+((NV−measured value)/(COP−NV)) for the case where a large measured value is predictive of poor outcome.

26. A method as in claim 2 wherein the RP is calculated using as inputs the parameters by a feature extraction mechanism that computes, as the measured value, the difference between the average value of select variables or ratios of select variables obtained at rest and during exercise in sub-maximal exercise bouts and applied to the formula RP=1+((measured value−NV)/(NV−COP)) for the case where a small measured value is predictive of poor outcome.

27. A method as in claim 2 wherein the RP is calculated using as inputs the parameters determined, in part, from known statistical values for the feature extraction mechanism that compute a measured value selected from the group consisting of the slope of the line of regression obtained from select data pairs obtain in sub-maximal exercise bouts and the difference between the average value of select variables or ratios of selected variables obtained in sub-maximal exercise bouts and applied to the formula RP=1+((measured value−NV)/(NV−COP)) for the case where a small measured value is predictive of poor outcome.

28. A method as in claim 2 wherein the measured MPI is located and displayed on a numeric axis that ranges from positive to negative values.

29. A method as in claim 18 wherein values from another functional classification system are juxtaposed onto the numeric axis as a reference.

30. A method as in claim 2 wherein the values of MPI for each unique test are plotted and the constituent parameters (Wₙ*RPₙ) of MPI are stacked to form a vertical bar, the height of which is the MPI value, and the bar annotated with the date of each test in a time sequential manner.
